(19) United States
(12) Patent Application Publication
(10) Pub. No.: US 2011/0218457 A1
(43) Pub. Date: Sep. 8, 2011

(54) DISPOSABLE VIEWABLE UTERUS CAVITY SUCTION TUBE

(75) Inventors: Xiongyi Song, Shanghai (CN); Kah-Ong Tan, Shanghai (CN)

(73) Assignee: OMNIVISION TECHNOLOGIES, INC., Santa Clara, CA (US)

(21) Appl. No.: 12/951,844

(22) Filed: Nov. 22, 2010

(30) Foreign Application Priority Data
Mar. 8, 2010 (CN) 201020125444.5

(51) Int. Cl. A61B 10/00

(52) U.S. Cl. 600/571

(57) ABSTRACT

A disposable viewable uterus cavity suction tube is used in conjunction with a negative pressure suction system and a display system. A suction opening is at the side wall of the uterus cavity suction tube. A separator is between the suction opening and the front end of the uterus cavity suction tube, which separates the uterus cavity suction tube into the suction cavity and the enclosed imaging cavity. An imaging head and an illumination device are in the imaging cavity. A see-through cover is at the tube wall above the imaging head and illumination device. The angle β between the central optical axis of the lens of the imaging head and the central normal of the suction opening is a sharp angle. The signal wire of the imaging head and illumination system are installed inside the tube wall. The characteristic of the disposable viewable uterus cavity suction tube is that the lens of the imaging head is a wide angle lens. By applying the aforementioned technical concept, the view angle of the disposable viewable uterus cavity suction tube can be larger than 80°, which allows the operator conveniently observing the whole image of the target.

### CROSS REFERENCE TO RELATED APPLICATIONS

The present application claims priority benefit under 35 U.S.C. §119(a) to Chinese application No. 201020125444.5 filed on Mar. 8, 2010 with the State Intellectual Property Office of the People's Republic of China.

### TECHNICAL FIELD

This disclosure relates to medical equipment, more specifically, relates to a disposable uterus cavity suction tube used in conjunction with a negative pressure suction system and a display system.

### BACKGROUND INFORMATION

The current disposable uterus cavity suction tube cannot be used to view the scene inside the uterus. It is operated based on the doctor's clinical experience. Thus it suffers from disadvantages of blind operation of uterus suction and large wound surface. It also easily induces infection and complication, which causes the patient suffering. Furthermore, due to the blind operation, the doctor relies on the touching of the tube to find the embryo. This precludes the operation until the embryo grows to certain size, and thus the pre-operation waiting time of the patient is largely extended.

For this reason, the applicant has designed a disposable viewable uterus cavity suction tube, see Chinese utility model patent publication number CN201139619Y dated 2008 Oct. 29. The publication discloses that the disposable viewable uterus cavity suction tube is equipped with an imaging head and an illumination device inside its imaging cavity. A see-through cover is at the tube wall above the imaging head and illumination device. During the surgery operation, the doctor can clearly observe the actual situation inside the patient's uterus cavity to perform a specific aimingful operation. However, using the aforementioned prior art technique, the distance to the target is only 2-3 mm. The viewable range is relatively small. The target ratio is relatively large, thus the operator cannot observe the whole image of the target.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 is the schematic diagram of disposable viewable uterus cavity suction tube in accordance with an embodiment. In the FIGURE, 1: suction cavity, 2: imaging cavity, 3: tube wall, 4: suction opening, 5: imaging head, 6: illumination device, 7: see-through cover, 8: separator, 9: counter.

### DETAILED DESCRIPTION

This disclosure is to overcome the drawback of the prior art technique, providing a disposable viewable uterus cavity suction tube having larger viewing range.

The technical concept of the invention is as follow. A disposable viewable uterus cavity suction tube is used in conjunction with a negative pressure suction system and a display system. A suction opening is at the side wall of the uterus cavity suction tube. A separator is between the suction opening and the front end of the uterus cavity suction tube, which separates the uterus cavity suction tube into the suction cavity and the enclosed imaging cavity. An imaging head and an illumination device are in the imaging cavity. A see-through cover is at the tube wall above the imaging head and illumination device. The angle β between the central optical axis of the lens of the imaging head and the central normal of the suction opening is a sharp angle (<90°). The signal wire of the imaging head and illumination system is installed inside the tube wall. The disposable viewable uterus cavity suction tube is characterized by the lens of the imaging head being a wide angle lens.

By applying the aforementioned technical concept, the view angle of the disposable viewable uterus cavity suction tube can be larger than 80°, which allows the operator conveniently observing the whole image of the target.

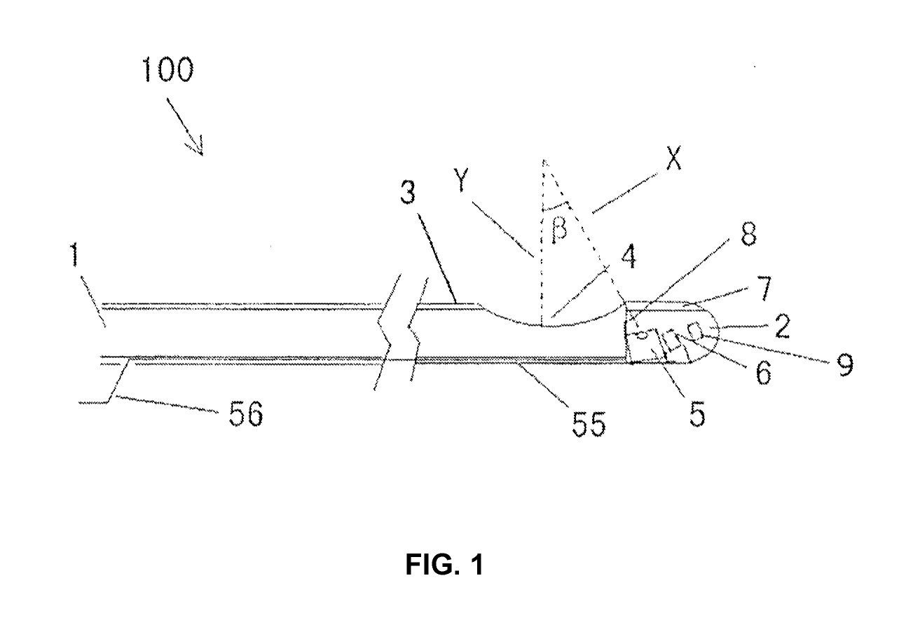

FIG. 1 illustrates a disposable viewable uterus cavity suction tube 100 in accordance with an embodiment. A side of a tube wall 3 of uterus cavity suction tube 100 is opened to form a suction opening 4. A separator 8 is between suction opening 4 and a front end of uterus cavity suction tube 100. Another end of uterus cavity suction tube is coupled with a negative pressure suction system. Separator 8 separates uterus cavity suction tube into a suction cavity 1 and an enclosed imaging cavity 2. The liquid and tissues drawn through suction opening 4 entirely enter suction cavity 1. An imaging head 5, an illumination device 6, and a power trigger counter 9 are inside imaging cavity 2. A see-through cover 7 is at the tube wall above imaging head 5 and illumination device 6. A lens of imaging head 5 is a wide angle lens. Furthermore, the see-through cover is preferably a concave lens, so the field of view (FOV) of the disposable viewable uterus cavity suction tube is larger than 80° and the operator can conveniently observe the whole image of the target. Counter 9 is coupled with a control unit which is disposed outside the disposable viewable uterus cavity suction tube. The control unit may be a single-chip processor or a programmable control device. The control unit is used for monitoring and changing the status word of counter 9. When the control unit determines the status word of the counter for power readout different from the preset status word, the control unit could make the imaging function of the disposable viewable uterus cavity suction tube ineffective by controlling the power supply of imaging head 5 or disconnecting the signal of the imaging head, to serve the purpose of one-time-use of the suction tube. This prevents repeated uses that cause infection. An angle β between a central optical axis X of imaging head 5 and a central normal Y is a sharp angle (<90°). Preferably, illumination device 6 comprises LED, see-through cover 7 is a transparent cover, angle β formed between central optical axis X of imaging head 5 and central normal Y of suction opening 4 is 0°≦β≦85°. Signal wire 55 of the imaging head and illumination device is installed inside tube wall 3 of the uterus cavity suction tube. After passing through and exiting from the tube wall, an end 56 of signal wire 55 is coupled with a display system. In a preferable embodiment: separator 8 is disposed next to suction opening 4; imaging head 5 is disposed next to separator 8; imaging head 5, illumination device 6, and counter 9 are integrated on a circuit board; the circuit board is slantedly fixed to tube wall 3 of the uterus cavity suction tube.

The imaging head of the embodiment is very tiny, thus it changes the structure of the current disposable viewable uterus cavity suction tube very little. Also, the imaging head, the illumination device, and the counter can be integrated on a circuit board forming an imaging module. It merely requires the implantation of the imaging module into the front end of the current disposable viewable uterus cavity suction tube, and changing the tube wall above the imaging head with a see-through material. Furthermore, only a few wires come out from the imaging module, no illumination optical fiber is required, and the output image signal can be directly connected to a display system. The imaging head is disposed next to the suction opening, and the optical axis of the imaging head and the central normal of the suction opening form a certain sharp angle, resulting in “what you see is what you suction”.

The embodiment is simple in structure and convenient in use. It merely adds a display system to the previous operation facility for uterus cavity and it is ready for use.

Although the description in this disclosure incorporates specific example embodiments, a person with ordinary skill in the art should understand that the disclosed utility model is not limited to the described example embodiments. The embodiments can be modified and changed without departing from the spirit and scope of the disclosure. It should be appreciated that the description of the specification is illustrative and not restrictive.

What is claimed is:

1. A disposable viewable uterus cavity suction tube used in conjunction with a negative pressure suction system and a display system, comprising:
a side wall of said uterus cavity suction tube having a suction opening;
a separator between said suction opening and a front end of said uterus cavity suction tube, said separator separating said uterus cavity suction tube into a suction cavity and an enclosed imaging cavity;
an imaging head and an illumination device in said imaging cavity;
a see-through cover at a tube wall of said uterus cavity suction tube above said imaging head and said illumination device; and
a signal wire of said imaging head and said illumination device installed inside said tube wall of said uterus cavity suction tube;
wherein an angle between a central optical axis of a lens of said imaging head and a central normal of said suction opening is a sharp angle, and
wherein said lens of said imaging head is a wide angle lens.

2. The disposable viewable uterus cavity suction tube of claim 1, wherein said see-through cover is a concave lens.

3. The disposable viewable uterus cavity suction tube of claim 2 further comprises a power triggered counter in said imaging cavity, said counter coupled with a control unit disposed outside said disposable viewable uterus cavity suction tube.

4. The disposable viewable uterus cavity suction tube of claim 3, wherein said control unit is one of single-chip processor and programmable control device.

5. The disposable viewable uterus cavity suction tube of claim 4, wherein said imaging head, said illumination device, and said counter are integrated on a circuit board.

6. The disposable viewable uterus cavity suction tube of claim 5, wherein said circuit board is slantedly fixed to said tube wall of said uterus cavity suction tube.

7. The disposable viewable uterus cavity suction tube of claim 5, wherein said illumination device comprises LED.

8. The disposable viewable uterus cavity suction tube of claim 1, wherein said separator is next to said suction opening, said imaging head is next to said separator.

9. The disposable viewable uterus cavity suction tube of claim 1, wherein said angle is 0°≦β≦85°.
